(19) United States
(12) Patent Application Publication
(10) Pub. No.: US 2025/0186721 A1
(43) Pub. Date: Jun. 12, 2025

(54) IMPROVEMENTS RELATING TO NASAL PIECES

(71) Applicant: Bespak Europe Limited, King's Lynn (GB)

(72) Inventor: Liset Rodriguez, King's Lynn (GB)

(21) Appl. No.: 18/843,187

(22) PCT Filed: Feb. 27, 2023
(86) PCT No.: PCT/EP2023/054804

§ 371 Date: Aug. 30, 2024

(30) Foreign Application Priority Data
Mar. 2, 2022 (GB) 2202890.6

(51) Int. Cl. A61M 15/08

(52) U.S. Cl. CPC A61M 15/08; A61M 2202/0007; A61M 2202/04; A61M 2207/00; A61M 2210/0618

(57) ABSTRACT

A nasal piece (1) comprises a body (2) having a tip (3) for insertion into a range of sizes of nostril, the body (2) comprising: a first flared section (10) that widens in a longitudinal direction away from the tip (3); a second flared section (11) that also widens in the longitudinal direction away from the tip (3); the first flared section (10) being more proximate to the tip (3) than the second flared section (11); the first flared section (10) defining a first abutment zone (20) for engaging in a first nostril having a first range of sizes; the second flared section (11) defining a second abutment zone (21) for engaging in a second nostril having a second range of sizes; wherein the first abutment zone (20) has a maximum flare angle that is greater than a maximum flare angle of the second abutment zone (21).

The present disclosure relates to nasal pieces. In addition, it relates to nasal sprays, nasal cannulas, nebulizers or nasal dilators comprising a nasal piece, as well as use of a nasal piece and a method of manufacturing a nasal piece.

### Background to the Disclosure

A nasal piece is an item or component of a device designed to engage in a nostril of a user. A nasal piece may be intended to permit the delivery of a substance into the nasal passages, for example when provided as part of a nasal spray, nasal cannula or nebulizer. A nasal piece may also (or in addition) be intended to open the nostril of a user, for example when provided as, or as part of, a nasal dilator.

The sizing of a nasal piece relative to the size of a user's nostril may, in many cases, be important to maximise the benefits of the item or device. For example, in cases where the nasal piece is used to deliver a substance into the nasal passages, the efficiency of the delivery of the substance may be affected by factors including the degree of sealing between the nasal piece and the nostril and the location of an outlet orifice of the nasal piece vis-à-vis the users' nasal cavity. In cases where the nasal piece is used to dilate the nostril, the efficiency and comfort of the dilation will be affected by factors including the size of the nasal piece vis-à-vis the user's nostril size.

Typically, nasal pieces are provided in a single size only for each device for reasons such as manufacturing economy. This may result in a decreased efficiency of substance delivery or nostril dilation for many users considering the wide range of nostril sizes of users.

### SUMMARY OF THE DISCLOSURE

An embodiment of the present disclosure provides a nasal piece comprising a body having a tip for insertion into a range of sizes of nostril, the body comprising:
a first flared section that widens in a longitudinal direction away from the tip;
a second flared section that also widens in the longitudinal direction away from the tip;
the first flared section being more proximate to the tip than the second flared section;
the first flared section defining a first abutment zone for engaging in a first nostril having a first range of sizes;
the second flared section defining a second abutment zone for engaging in a second nostril having a second range of sizes;
wherein the first abutment zone has a maximum flare angle that is greater than a maximum flare angle of the second abutment zone.

Advantageously, the nasal piece of the present disclosure may provide improved sealing in a wide range of sizes of nostril. The provision of separate first and second flared sections may permit a single nasal piece to be suitable for use by both adult and paediatric users.

For example, the first flared section may be configured for use by paediatric users or a sub-range of paediatric users. In some examples the first flared section may be configured for use by users aged 0-5 years.

For example, the second flared section may be configured for use by adult users (and optionally also older paediatric users). In some examples the second flared section may be configured for use by users aged 5 years and above, optionally 18 years and above.

Configuring the first abutment zone to have a greater maximum flare angle than the second abutment zone may be advantageous in better configuring the nasal piece for both paediatric and adult users. In particular, the variation in nostril diameter for paediatric users, especially those under 7 years of age, is relatively large. For this reason it has been found that providing a relatively large maximum flare angle for the first abutment zone, and hence a steeper slope to the nasal piece in this area, is beneficial in accommodating a large variation in nostril diameter without leading to over-insertion.

Advantageously, the nasal piece of the present disclosure may provide a single nasal piece that may be used by many, and potentially all, ages of user. The nasal piece may also at the same time increase the comfort and efficiency of the nasal piece by better configuring the location of abutment of the nasal piece with the nostril for a range of users having a range of nostril sizes. In addition, the nasal piece may be better suited for use by both male and female users who may, typically, have differing ranges of nostril sizes.

Advantageously, the nasal piece of the present disclosure may reduce the cost and complexity of manufacture of devices by avoiding (or at least reducing) the need to provide a range of separate nasal pieces of differing sizes for a single device.

The first abutment zone may comprise a whole or a part of the first flared section. The second abutment zone may comprise a whole or a part of the second flared section.

The first flared section and/or the second flared section of the nasal piece may have circular symmetry about the longitudinal axis of the nasal piece. Alternatively, the first flared section and/or the second flared section of the nasal piece may not have circular symmetry about the longitudinal axis of the nasal piece, e.g. an external diameter of the first flared section and/or the second flared section may differ front-to-back from side-side.

The maximum flare angle of the first abutment zone may be 35° to 65°, preferably 40° to 60°, more preferably 58° and/or the maximum flare angle of the second abutment zone may be 10° to 30°, preferably 15° to 25°, more preferably 18°.

In some examples, a flare angle of the first abutment zone may be constant along a longitudinal length of the first abutment zone. For example, the first abutment zone may be provided by a frusto-conical section of the body. The constant flare angle of the first abutment zone may be, for example, 35° to 65°, preferably 40° to 60°, more preferably 58°.

In alternative examples, a flare angle of the first abutment zone may vary along a longitudinal length of the first abutment zone. For example, the first abutment zone may be provided by a concavely-shaped (as viewed from an exterior of the nasal piece) section of the body or alternatively a series of frusto-conical sections. The flare angle may vary, for example, from a minimum flare angle of 15° to 20° to the maximum flare angle of 35° to 65°, preferably 40° to 60°, more preferably 58°. In examples where the first abutment zone is concavely-shaped, the first abutment zone may have a constant or variable radius of curvature along its longitudinal length.

In some examples, a flare angle of the second abutment zone may be constant along a longitudinal length of the second abutment zone. For example, the second abutment zone may be provided by a frusto-conical section of the body. The constant flare angle of the second abutment zone may be, for example, 10° to 30°, preferably 15° to 25°, more preferably 18°.

In alternative examples, a flare angle of the second abutment zone may vary along a longitudinal length of the second abutment zone. For example, the second abutment zone may be provided by a concavely-shaped (as viewed from an exterior of the nasal piece) section of the body or alternatively a series of frusto-conical sections. The flare angle may, for example, vary from a minimum flare angle of 5° to 10° to the maximum flare angle of 10° to 30°, preferably 15° to 25°, more preferably 18°. In examples where the second abutment zone is concavely-shaped, the second abutment zone may have a constant or variable radius of curvature along its longitudinal length.

Preferably, a longitudinal length of the first abutment zone and/or the first flared section is shorter than a longitudinal length of the second abutment zone and/or the second flared section. Beneficially, this may help to prevent over-insertion of the tip of the nasal piece into the nostrils of paediatric users, especially those under 7 years of age.

In some examples the first abutment zone extends at least between locations that are 5.0 mm to 7.0 mm longitudinally from the tip. This has been found to be a beneficial location for engaging the nostril of paediatric users and providing good positioning of the tip of the nasal piece vis-à-vis the nasal valve of the nasal cavity.

In some examples the second abutment zone extends at least between locations that are 12.0 mm to 15.0 mm longitudinally from the tip; optionally at least between locations that are 12.0 mm to 20.0 mm longitudinally from the tip; optionally at least between locations that are 10.0 mm to 20.0 mm longitudinally from the tip. This has been found to be a beneficial location for engaging the nostril of adult users providing and good positioning of the tip of the nasal piece vis-à-vis the nasal valve of the nasal cavity.

Preferably, the second flared section is wider than the first flared section.

In some examples an external diameter of the first abutment zone at its end nearest the tip may have a diameter of 6.60 to 7.50 mm, optionally of 6.90 to 7.20 mm, optionally of 7.09 mm; and/or an external diameter of the first abutment zone at its end furthest from the tip may have a diameter of 9.50 to 11.00 mm, optionally of 9.75 to 10.50 mm, optionally of 10.43 mm.

In some examples an external diameter of the second abutment zone at its end nearest the tip has a diameter of 10.50 to 11.50 mm, optionally of 10.70 to 11.00 mm, optionally of 10.70 to 10.90 mm, optionally of 10.85 mm or 10.96 mm; and/or an external diameter of the second abutment zone at its end furthest from the tip has a diameter of 11.00 to 20.00 mm, optionally 11.00 to 16.00 mm, optionally 11.00 to 15.00 mm, optionally of 11.75 to 15.00 mm, optionally of 14.00 to 15.00 mm, optionally of 11.75 mm or 14.75 mm.

Preferably, the body comprises a shoulder or convex section that separates at least a portion of the first abutment zone from at least a portion of the second abutment zone. In some examples the shoulder or convex section may overlap partially with the first abutment zone and/or the second abutment zone. In some examples the shoulder or convex section may overlap wholly or partially with the first flared section and/or the second flared section.

Provision of the shoulder or convex section of the nasal piece may beneficially allow for a more compact nasal piece, in particular in shortening the longitudinal length of the nasal piece. In addition, the provision of the shoulder or convex section permits the wider, second abutment zone to be positioned closer to the tip of the nasal piece than would be the case with a nasal piece having a single taper. This may help to prevent over-insertion of the tip of the nasal piece into the nostrils of users.

In some examples the shoulder or convex section of the body is located between 5.0 mm and 10.0 mm longitudinally from the tip.

In some examples the first flared section comprises a frusto-conical and/or a concave section and/or a convex section and/or the second section comprises a frusto-conical and/or concave section and/or a convex section. Either or both of the first abutment zone and the second abutment zone may be in the form of one or more frusto-conical sections. Either or both of the first abutment zone and the second abutment zone may be in the form of one or more concavely-shaped sections. Either or both of the first abutment zone and the second abutment zone may be in the form of one or more convexly-shaped sections. Frusto-conical section(s) and concavely-shaped section(s) and convexly-shaped section(s) may be combined in the same nasal piece.

In some examples a longitudinal axis of the first flared section is coincident with a longitudinal axis of the second flared section.

In some examples the body further comprises a tip section that comprises the tip of the body and extends from the tip to the first flared section, optionally wherein the tip section is convex.

In some examples, for example where the nasal piece is for use as part of a nasal spray, the body further comprises one or more shoulders for engagement by one or more fingers, the one or more shoulders being located further from the tip than the second flared section.

Another embodiment of the present disclosure provides a nasal spray, nasal cannula, nebulizer or nasal dilator comprising a nasal piece as described in the above embodiment. In the case of a nasal spray the device may, for example, be of a type used for nasal inhalation of a medicament.

Another embodiment of the present disclosure is use of a nasal piece of the first embodiment above, wherein in a first mode of use the nasal piece is inserted into a first nostril having a first range of sizes such that the first nostril is abutted against the first abutment zone of the first flared section; and in a second mode of use the nasal piece is inserted into a second nostril having a second range of sizes such that the second nostril is abutted against the second abutment zone of the second flared section.

The first nostril may be a nostril of a paediatric user and the second nostril may be a nostril of an adult user.

Another embodiment of the present disclosure provides a method of manufacturing a nasal piece of the first embodiment above, the method comprising forming the body of the nasal piece to have the tip, the first flared section that widens in a longitudinal direction away from the tip and the second flared section that also widens in the longitudinal direction away from the tip, wherein the first flared section is more proximate to the tip than the second flared section, and the first flared section defines the first abutment zone for engaging in the first nostril having the first range of sizes, and the second flared section defines the second abutment zone for engaging in the second nostril having the second range of sizes, wherein the first abutment zone has a maximum flare angle that is greater than a maximum flare angle of the second abutment zone.

In some examples the nasal piece is formed as a single piece; and optionally formed by moulding, preferably injection moulding.

### BRIEF DESCRIPTION OF THE DRAWINGS

One or more embodiments of the disclosure will now be described, by way of example only, with reference to the accompanying drawings in which:

FIG. 1 is a front elevation view of a nasal piece according to the present disclosure;

FIG. 2 is a plan view from above of the nasal piece of FIG. 1;

FIG. 3 is a perspective view from above of the nasal piece of FIG. 1;

FIG. 4 is a side elevation view of the nasal piece of FIG. 1 depicting flare angles of the nasal piece;

FIGS. 5 to 7 are side elevation views of the nasal piece of FIG. 1 depicting dimensions of the nasal piece;

FIG. 8A is a partial front elevation view of a nasal piece according to the present invention comprising concavely-shaped abutment zones;

FIG. 8B is a partial front elevation view of a nasal piece according to the present invention comprising frusto-conical abutment zones; and

FIG. 9 is a front elevation view of a nasal spray comprising the nasal piece of FIG. 1.

### DETAILED DESCRIPTION

Unless defined otherwise, all technical and scientific terms used in this specification have the same meaning as is commonly understood by the reader skilled in the art to which the claimed subject matter belongs. It is to be understood that the foregoing summary of the disclosure and the following examples are exemplary and explanatory only and are not restrictive of any subject matter claimed.

The following description is directed to embodiments of the disclosure. The description of the embodiments is not meant to include all the possible embodiments of the disclosure that are claimed in the appended claims. Many modifications, improvements and equivalents which are not explicitly recited in the following embodiments may fall within the scope of the appended claims. Features described as part of one embodiment may be combined with features of one or more other embodiments unless the context clearly requires otherwise.

In this specification, the use of the singular includes the plural unless the context clearly dictates otherwise. In this application, the use of “and/or” means “and” and “or” unless stated otherwise.

In this specification, the term “flare angle” means for a point on the external surface of the nasal piece, the angle between the longitudinal axis of the nasal piece and the tangent to the external surface at that point—as shown, by way of example, in FIG. 4.

The present disclosure relates generally to nasal pieces. In addition, it relates to nasal sprays, nasal cannulas, nebulizers or nasal dilators comprising a nasal piece, as well as use of a nasal piece and a method of manufacturing a nasal piece.

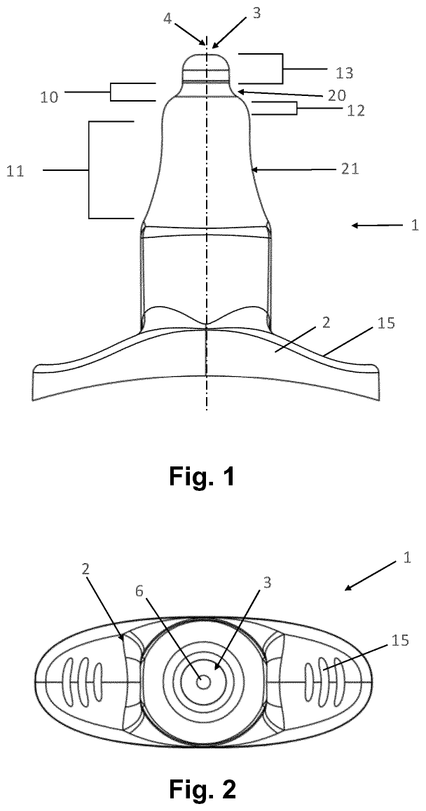

FIGS. 1 to 7 show a nasal piece 1 comprising a body 2 having a tip 3 for insertion into a nostril of a user. As shown in FIG. 1, the body 2 comprises a first flared section 10 that widens in a longitudinal direction away from the tip 3 and a second flared section 11 that also widens in the longitudinal direction away from the tip 3. The first flared section 10 is more proximate to the tip 3 than the second flared section 11.

The first flared section 10 defines a first abutment zone 20 for engaging in a first nostril having a first range of sizes.

The second flared section 11 define a second abutment zone 21 for engaging in a second nostril having a second range of sizes.

The first abutment zone 20 has a maximum flare angle that is greater than a maximum flare angle of the second abutment zone 21.

The maximum flare angle of the first abutment zone 20 may be 35° to 65°, preferably 40° to 60°. More preferably, as shown in FIG. 4, the maximum flare angle of the first abutment zone 20 may be 58°.

The maximum flare angle of the second abutment zone 21 may be 10° to 30°, preferably 15° to 25°. More preferably, as shown in FIG. 4, the maximum flare angle of the second abutment zone 21 may be 18°.

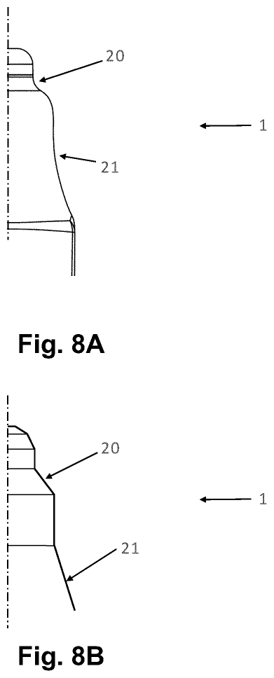

The flare angle of the first abutment zone 20 may be constant along a longitudinal length of the first abutment zone 20. This may be the case, for example, where the first flared section 10 has a frusto-conical form as shown, by way of example, in FIG. 8B. For example, the constant flare angle of the first abutment zone 20 may be 35° to 65°, preferably 40° to 60°, more preferably 58°.

Alternatively, the flare angle of the first abutment zone 20 may vary along the longitudinal length of the first abutment zone 20. This may be the case, for example, where the first flared section 10 has a concavely-shaped form as shown, by way of example, in FIG. 8A. For example, the flare angle may vary from a minimum flare angle of 15° to 20° to the maximum flare angle of 35° to 65°, preferably 40° to 60°, more preferably 58°.

Similarly, the flare angle of the second abutment zone 21 may be constant along the longitudinal length of the second abutment zone 21. This may be the case, for example, where the second flared section 11 has a frusto-conical form as shown, by way of example, in FIG. 8B. For example, the constant flare angle of the second abutment zone 21 may be 10° to 30°, preferably 15° to 25°, more preferably 18°.

Alternatively, the flare angle of the second abutment zone 21 may vary along the longitudinal length of the second abutment zone 21. This may be the case, for example, where the second flared section 11 has a concavely-shaped form as shown, by way of example, in FIG. 8A. For example, the flare angle may vary from a minimum flare angle of 5° to 10° to the maximum flare angle of 10° to 30°, preferably 15° to 25°, more preferably 18°.

The longitudinal length of the first abutment zone 20 and/or the first flared section 10 may be shorter than the longitudinal length of the second abutment zone 21 and/or the second flared section 11.

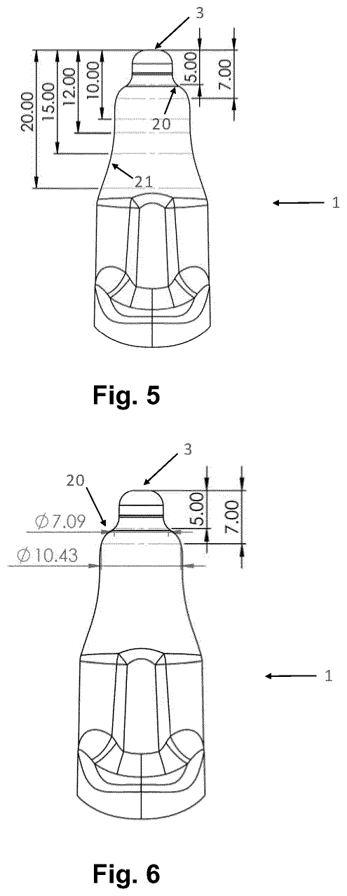

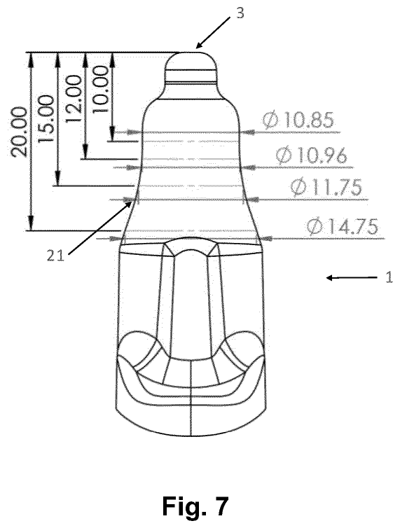

The first abutment zone 20 may extend at least between locations that are 5.0 mm to 7.0 mm longitudinally from the tip 3, as shown by way of example in FIGS. 5 and 6. The second abutment zone 21 may extend at least between locations that are 12.0 mm to 15.0 mm longitudinally from the tip 3; optionally at least between locations that are 12.0 mm to 20.0 mm longitudinally from the tip 3; optionally at least between locations that are 10.0 mm to 20.0 mm longitudinally from the tip 3, as shown by way of example in FIGS. 5 and 7.

The second flared section 11 may be wider than the first flared section 10.

An external diameter of the first abutment zone 20 at its end nearest the tip may have a diameter of 6.60 to 7.50 mm, optionally of 6.90 to 7.20 mm, optionally of 7.09 mm. The measurement may be, for example, at 5.00 mm longitudinally from the tip 3 as shown in the example of FIG. 6. An external diameter of the first abutment zone 20 at its end furthest from the tip 3 may have a diameter of 9.50 to 11.00 mm, optionally of 9.75 to 10.50 mm, optionally of 10.43 mm. The measurement may be, for example, at 7.00 mm longitudinally from the tip 3 as shown in the example of FIG. 6.

An external diameter of the second abutment zone 21 at its end nearest the tip may have a diameter of 10.50 to 11.50 mm, optionally of 10.70 to 11.00 mm, optionally of 10.70 to 10.90 mm, optionally of 10.85 mm or 10.96 mm. The measurement may be, for example, at 7.00 mm or 10.00 mm longitudinally from the tip 3 as shown in the example of FIG. 7. An external diameter of the second abutment zone 21 at its end furthest from the tip 3 may have a diameter of 11.00 to 20.00 mm, optionally 11.00 to 16.00 mm, optionally 11.00 to 15.00 mm, optionally of 11.75 to 15.00 mm, optionally of 14.00 to 15.00 mm, optionally of 11.75 mm or 14.75 mm. The measurement may be, for example, at 15.00 mm or 20.00 mm longitudinally from the tip 3 as shown in the example of FIG. 7.

The body 2 may comprise a shoulder or convex section 12 that separates at least a portion of the first abutment zone 20 from at least a portion of the second abutment zone 21. The shoulder or convex section 12 of the body 2 may be located between 5.0 mm and 10.0 mm longitudinally from the tip 3. The shoulder or convex section 12 may overlap partially with the first abutment zone 20 and/or the second abutment zone 21. Additionally or alternatively the shoulder or convex section 12 may overlap wholly or partially with the first flared section 10 and/or the second flared section 11.

The first flared section 10 may comprise a frusto-conical and/or a concave section and/or a convexly-shaped section and/or the second flared section 11 may comprise a frusto-conical and/or concave section and/or a convexly-shaped section.

The longitudinal axis of the first flared section 10 may be coincident with the longitudinal axis of the second flared section 11, as in the example of FIG. 1 where the longitudinal axis is labelled 4.

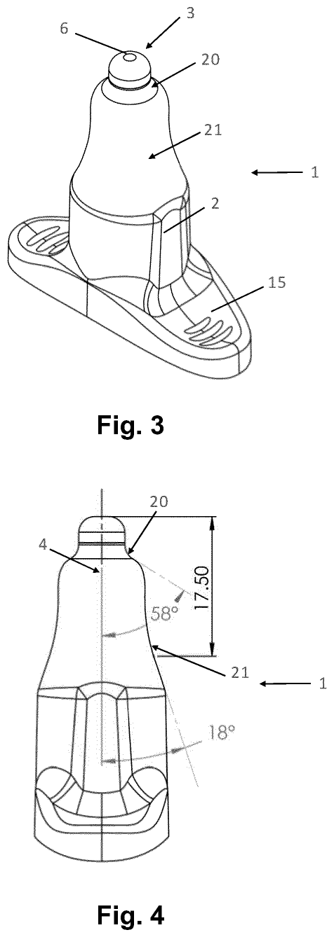

The body 2 may further comprise a tip section 13 that comprises the tip 3 of the body 2 and extends from the tip 3 to the first flared section 10, optionally wherein the tip section 13 is convex as shown in FIGS. 1 and 3. An outlet orifice 6 may be provided in the tip section 13, optionally at the apex of the tip 3, as shown in FIGS. 2 and 3.

The body 2 may further comprise, as shown in FIGS. 1 and 3, one or more shoulders 15 for engagement by one or more fingers, the one or more shoulders 15 being located further from the tip 3 than the second flared section 11.

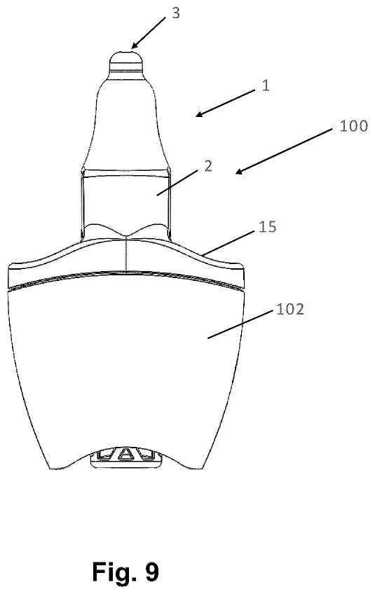

A nasal spray, nasal cannula, nebulizer or nasal dilator may be provided that comprises a nasal piece 1 as described above. An example of a nasal spray 100 is shown in FIG. 9, wherein the nasal piece 1 is mounted on top of a body 102 of the nasal spray 100. In use, a source of product, for example a medicament to be delivered nasally, may be housed within the body 102.

The nasal piece 1 of the present disclosure may be used in a first mode of use wherein the nasal piece 1 is inserted into a first nostril having a first range of sizes such that the first nostril is abutted against the first abutment zone 20 of the first flared section 10 and in a second mode of use wherein the nasal piece 1 is inserted into a second nostril having a second range of sizes such that the second nostril is abutted against the second abutment zone 21 of the second flared section 11. For example, the first nostril may be a nostril of a paediatric user and the second nostril may be a nostril of an adult user.

The nasal piece 1 of the present disclosure may be manufactured so as to be formed as a single piece. The nasal piece 1 may be, for example, formed by moulding, preferably injection moulding.

### INDUSTRIAL APPLICABILITY

The present disclosure finds application in the provision of nasal pieces for devices, for example nasal sprays, nasal cannulas, and nebulizers or nasal dilators comprising a nasal piece.

It is to be understood that at least some of the figures and descriptions of the disclosure have been simplified to focus on elements that are relevant for a clear understanding of the disclosure, while eliminating, for purposes of clarity, other elements that the reader skilled in the art will appreciate may also be required. Because such elements are well known to the reader skilled in the art, and because they do not necessarily facilitate a better 10 understanding of the disclosure, a description of such elements is not provided herein.

1. A nasal piece comprising a body having a tip for insertion into a range of sizes of nostril, the body comprising:
a first flared section that widens in a longitudinal direction away from the tip;
a second flared section that also widens in the longitudinal direction away from the tip;
the first flared section being more proximate to the tip than the second flared section;
the first flared section defining a first abutment zone for engaging in a first nostril having a first range of sizes;
the second flared section defining a second abutment zone for engaging in a second nostril having a second range of sizes;
wherein the first abutment zone has a maximum flare angle that is greater than a maximum flare angle of the second abutment zone.

2. A nasal piece as claimed in claim 1, wherein the maximum flare angle of the first abutment zone is 35° to 65°, preferably 40° to 60°, more preferably 58° and/or the maximum flare angle of the second abutment zone is 10° to 30°, preferably 15° to 25°, more preferably 18°.

3. A nasal piece as claimed in claim 1, wherein a flare angle of the first abutment zone is constant along a longitudinal length of the first abutment zone; and optionally wherein the flare angle of the first abutment zone is 35° to 65°, preferably 40° to 60°, more preferably 58°.

4. A nasal piece as claimed in claim 1, wherein a flare angle of the first abutment zone varies along a longitudinal length of the first abutment zone; and optionally wherein the flare angle varies from a minimum flare angle of 15° to 20° to the maximum flare angle of 35° to 65°, preferably 40° to 60°, more preferably 58°.

5. A nasal piece as claimed in claim 1, wherein a flare angle of the second abutment zone is constant along a longitudinal length of the second abutment zone; and optionally wherein the flare angle of the second abutment zone is 10° to 30°, preferably 15° to 25°, more preferably 18°.

6. A nasal piece as claimed in claim 1, wherein a flare angle of the second abutment zone varies along a longitudinal length of the second abutment zone; and optionally wherein the flare angle varies from a minimum flare angle of 5° to 10° to the maximum flare angle of 10° to 30°, preferably 15° to 25°, more preferably 18°.

7. A nasal piece as claimed in claim 1, wherein a longitudinal length of the first abutment zone and/or the first flared section is shorter than a longitudinal length of the second abutment zone and/or the second flared section.

8. A nasal piece as claimed in claim 1, wherein the first abutment zone extends at least between locations that are 5.0 mm to 7.0 mm longitudinally from the tip.

9. A nasal piece as claimed in claim 1, wherein the second abutment zone extends at least between locations that are 12.0 mm to 15.0 mm longitudinally from the tip; optionally at least between locations that are 12.0 mm to 20.0 mm longitudinally from the tip; optionally at least between locations that are 10.0 mm to 20.0 mm longitudinally from the tip.

10. A nasal piece as claimed in claim 1, wherein the second flared section is wider than the first flared section.

11. A nasal piece as claimed in claim 1, wherein an external diameter of the first abutment zone at its end nearest the tip has a diameter of 6.60 to 7.50 mm, optionally of 6.90 to 7.20 mm, optionally of 7.09 mm; and/or an external diameter of the first abutment zone at its end furthest from the tip has a diameter of 9.50 to 11.00 mm, optionally of 9.75 to 10.50 mm, optionally of 10.43 mm.

12. A nasal piece as claimed in claim 1, wherein an external diameter of the second abutment zone at its end nearest the tip has a diameter of 10.50 to 11.50 mm, optionally of 10.70 to 11.00 mm, optionally of 10.70 to 10.90 mm, optionally of 10.85 mm or 10.96 mm; and/or an external diameter of the second abutment zone at its end furthest from the tip has a diameter of 11.00 to 20.00 mm, optionally 11.00 to 16.00 mm, optionally 11.00 to 15.00 mm, optionally of 11.75 to 15.00 mm, optionally of 14.00 to 15.00 mm, optionally of 11.75 mm or 14.75 mm.

13. A nasal piece as claimed in claim 1, wherein the body comprises a shoulder or convex section that separates at least a portion of the first abutment zone from at least a portion of the second abutment zone.

14. A nasal piece as claimed in claim 13, wherein the shoulder or convex section of the body is located between 5.0 mm and 10.0 mm longitudinally from the tip.

15. A nasal piece as claimed in claim 1, wherein the first flared section comprises a frusto-conical and/or a concave section and/or a convex section and/or the second section comprises a frusto-conical and/or concave section and/or a convex section.

16. A nasal piece as claimed in claim 1, wherein a longitudinal axis of the first flared section is coincident with a longitudinal axis of the second flared section.

17. A nasal piece as claimed in claim 1, wherein the body further comprises a tip section that comprises the tip of the body and extends from the tip to the first flared section, optionally wherein the tip section is convex.

18. A nasal piece as claimed in claim 1, wherein the body further comprises one or more shoulders for engagement by one or more fingers, the one or more shoulders being located further from the tip than the second flared section.

19. A nasal spray, nasal cannula, nebulizer or nasal dilator comprising a nasal piece as claimed in claim 1.

20. A method of using the nasal piece as claimed in claim 1, comprising, in a first mode of use, inserting the nasal piece is inserted into a first nostril having a first range of sizes such that the first nostril is abutted against the first abutment zone of the first flared section; and, in a second mode of use, inserting the nasal piece into a second nostril having a second range of sizes such that the second nostril is abutted against the second abutment zone of the second flared section.

21. Use of a nasal piece as claimed in claim 20, wherein the first nostril is a nostril of a paediatric user and the second nostril is a nostril of an adult user.

22. A method of manufacturing the nasal piece as claimed in claim 1, comprising forming the body of the nasal piece to have the tip, the first flared section that widens in a longitudinal direction away from the tip and the second flared section that also widens in the longitudinal direction away from the tip, wherein the first flared section is more proximate to the tip than the second flared section, and the first flared section defines the first abutment zone for engaging in the first nostril having the first range of sizes, and the second flared section defines the second abutment zone for engaging in the second nostril having the second range of sizes, wherein the first abutment zone has a maximum flare angle that is greater than a maximum flare angle of the second abutment zone.

23. The method of claim 22, wherein the nasal piece is formed as a single piece; and optionally formed by moulding, preferably injection moulding.
